(19) United States
(12) Patent Application Publication
(10) Pub. No.: US 2015/0094635 A1
(43) Pub. Date: Apr. 2, 2015

(54) Arm Restricting Bandage

(71) Applicants: Barbara Keller, Kirkland, WA (US); Brian Scott Keller, Kirkland, WA (US)

(72) Inventors: Barbara Keller, Kirkland, WA (US); Brian Scott Keller, Kirkland, WA (US)

(21) Appl. No.: 14/043,956

(22) Filed: Oct. 2, 2013

(51) Int. Cl. A61F 5/05

(52) U.S. Cl. CPC A61F 5/05
USPC 602/20

(57) ABSTRACT

A bandage for restricting movement of an arm of a person is disclosed. At least one body panel formed of an elastic breathable material, where the at least one body panel has a left end, a right end and a bottom end, where the left end has a first closure mechanism, where the right end has a second closure mechanism. The at least one body panel is configured to wrap around a torso of the person, where the first closure mechanism and the second closure mechanism connects to each other to hold the at least one body panel onto the torso of the person. A sleeve formed of an elastic breathable material coupled to the bottom end of the at least one body panel, where the sleeve is configured to receive an arm of the person, where the arm is held in place by the sleeve.

### FIELD OF THE INVENTION

The present invention relates to an arm restricting bandage system. More particularly a person is able to utilize the arm restricting bandage to prevent the person from mobilizing his or her arm while utilizing the body for stabilization.

### BACKGROUND OF THE INVENTION

Oftentimes when human beings are active such as participating in sports or in normal activities they may injure themselves. They may injure themselves by fracturing or breaking their arms, legs or other parts of their bodies. In order to enable the arms, legs or other parts of their bodies to heal a cast may be used to place the arms or legs into for a period of time. In some cases, a sling may be the best possible device that may be used to help the arm heal. In order to heal arms and/or legs a cast may be used to immobilize the arms and/or legs for a period of time. In certain cases, a sling may be the best possible device used to help the arm heal when casting cannot be performed.

Slings are good for immobilizing arms for a short period of time while still making it possible for the person to move her arm. However, the problem with the sling is that it has a tendency to pull on the neck of the wearer of the sling and may take the neck out of alignment which causes more problems for the wearer. Also, it would be difficult for a person to conduct normal activities with a sling, such as writing or using utensils to eat food. Further, another problem with the sling is that it allows the arm to move away from the body, raise the arm overhead so children can simply move the arm out of the sling easily making it difficult for a child to use the sling properly.

Therefore, there is a need for a device that enables a person to safely and simply immobilize a portion of his or her body that doesn't cause his or her neck to be out of alignment.

### SUMMARY OF THE INVENTION

The present invention has been accomplished in view of the above-mentioned technical background, and it is an object of the present invention to provide an arm restricting bandage that immobilizes at least one arm.

In one embodiment of the invention, a bandage for restricting movement of an arm of a person is disclosed. At least one body panel is formed of an elastic breathable material, where the at least one body panel has a left end, a right end and a bottom end, where the left end has a first closure mechanism, where the right end has a second closure mechanism, The at least one body pan& is configured to wrap around a torso of the person, where the first closure mechanism and the second closure mechanism connects to each other to hold the at least one body panel onto the torso of the person. A sleeve formed of elastic breathable material coupled to the bottom end of the at least one body panel, where the sleeve is configured to receive an arm of the person, where the arm is held in place by the sleeve.

In another embodiment of the invention, a bandage for restricting movement of an arm of a person is disclosed. A plurality of body panels formed of an elastic breathable material, wherein each of the plurality of body panels have a left end, a right end and a bottom end, wherein the left end and the right end have fastening members to connect the left end to the right end. The plurality of body panels are configured to wrap around a torso of the person, wherein the fastening members hold the plurality of body panels onto the torso of the person. A sleeve is formed of an elastic breathable material coupled to the bottom end of one of the plurality of body panels, wherein the sleeve is configured to receive an arras of the person, wherein the arm is held in place by the sleeve.

### BRIEF DESCRIPTION OF THE DRAWINGS

These and other advantages of the present invention will become more apparent as the following description is read in conjunction with the accompanying drawings, wherein:

FIG. 1A is a perspective view of an arm restricting bandage laid open in accordance with the invention;

FIG. 1B is a perspective view of the arm restricting bandage of FIG. 1A in a overlapping position of the loop material disposed over the vertical strip in accordance with the invention;

FIG. 1C is an embodiment of the arm restricting bandage of FIG. 1B wrapped around a torso of a person in accordance with the invention;

FIG. 1D is an embodiment of the arm restricting bandage of FIG. 1B showing a back portion wrapped around a torso of a person in accordance with the invention;

FIG. 2A is a perspective view of another arm restricting bandage in a laid open position in accordance with the invention;

FIG. 2B is a perspective view of the arm restricting bandage of FIG. 2A in an overlapping position of the loop material disposed over the vertical strip in accordance with the invention;

FIG. 2C is an embodiment of the arm restricting bandage of FIG. 2B wrapped around a torso of a person in accordance with the invention; and

FIG. 2D is an embodiment of the arm restricting bandage of FIG. 2B showing a back portion wrapped around a torso of a person in accordance with the invention.

### DETAILED DESCRIPTION OF THE INVENTION

The presently preferred embodiments of the invention are described with reference to the drawings, where like components are identified with the same numerals. The descriptions of the preferred embodiments are exemplary and are not intended to limit the scope of the invention.

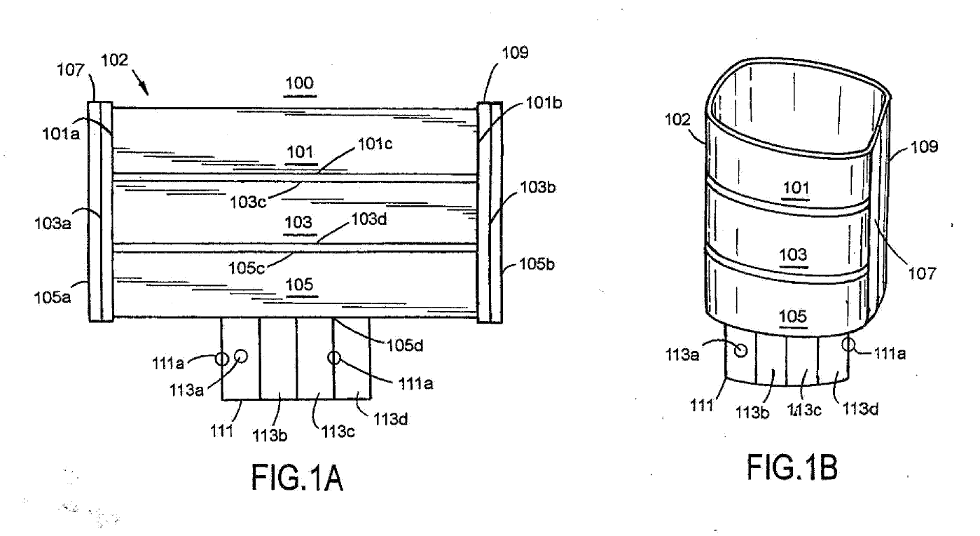

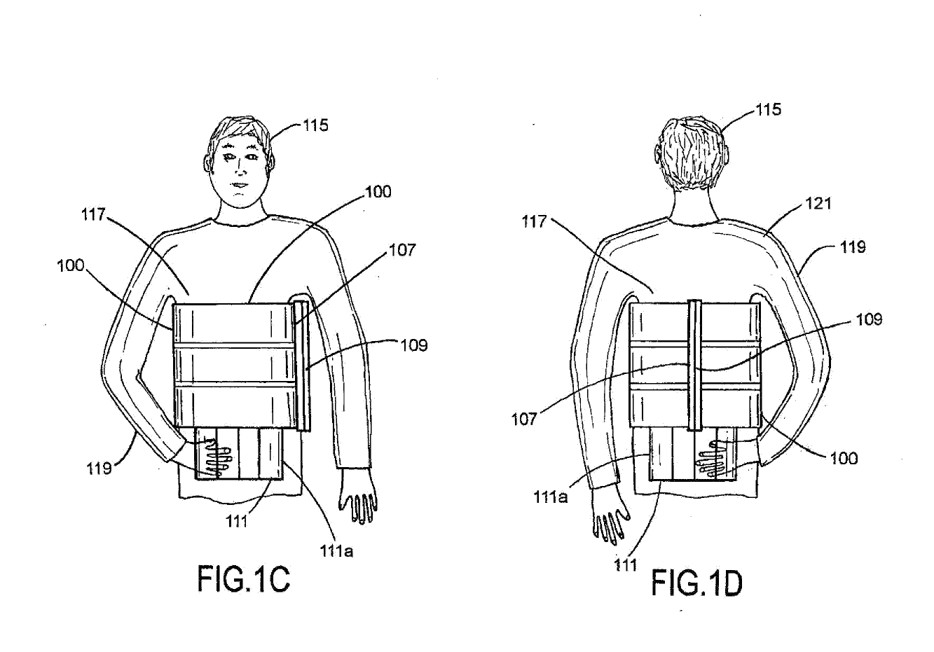

FIG. 1A is a perspective view of an arm restricting bandage laid open. The arm restricting bandage 100 has a main body panel 102 that is made of a first body panel 101, second body panel 103 and a third body panel 105. Even though there are three body panels 101, 103 and 105 utilized in this invention there may be 1, 2, 4, 5, 10 up to 100 or more body panels that make up the main body panel 102. The first body panel 101, second body panel 103 and the third body panel 105 have a generally elongated rectangular shape when they are laid open as shown. In another embodiment of the invention, the first body panel 101, second body panel 103 and third body panel 105 may have any shape such as a square shape, circular shape, triangular shape, elliptical shape or any type of shape. The main body panel 102 including first body panel 101, second body panel 103 and third body panel 105 may be made of any suitable elastic or non-elastic, breathable material such as an ACE bandage. ACE is a registered trademark of 3M Corporation having corporate headquarters in St. Paul, Minn. Main body panel 102, first body panel 101, second body panel 103 and third body panel 105 are used for body core stabilization. Core stabilization refers to the trunk of the body core being strong and able to support the rest of the muscles as they move. If the core is not stable because the muscles are weak, then one may lose balance more easily. The main muscles involved in core stability are the inner core muscles of the abdomen and pelvis. These muscles mostly support the spine and body while other muscles do the work of moving the rest of the body.

First body panel 101, second body panel 103 and third body panel 105 have a length in the range of 12-40 inches and a width in the range of 5-20 inches. The first body panel 101 has a left end 101a, a right end 101b, and a bottom end 101c. Second body panel 103 has a left end 103a, a right end 103b, a top end 103c and a bottom end 103d. Bottom end 101d is connected to the top end 103c by stitching these two ends together. Even though stitching is utilized to connect the bottom end 101d to the top end 103c any other method of connecting these ends together such as a hook/loop feature may be utilized. Third body panel 105 has a left end 105a, a right end 105b, a top end 105c and a bottom end 105d. Bottom end 103d is connected to the top end 105c by stitching these two ends together. Left end 101a, left end 103a and left end 105a all include a vertical strip 107. Right end 101b, right end 103b and right end 105b all include a loop material 109. Even though stitching is utilized to connect the bottom end 103d to the top end 105c any other method of connecting these ends together such as a hook/loop feature may be utilized.

A sleeve 111 is connected to the bottom end 105d where the sleeve 111 is stitched to the bottom end 105d of the third body panel 105. The sleeve 111 has an opening 111a that goes through the entire sleeve 111 where a person can insert his or her arm consisting of his or her hands, wrist and forearm through the sleeve 111. Opening 111a has a range of 4-15 inches circumferentially from the bottom end 105d to fit the hand, wrist and the forearm. This sleeve 111 includes a first portion 113a, second portion 113b, third portion 113c and fourth portion 113d elastic breathable bandages that are circularly connected to the bottom end 105d to form the opening 111a. First portion 113a, second portion 113b, third portion 113c and fourth portion 113d have a total length in a range of 6-16 inches to fit the hand, wrist and the forearm. The person can insert her arm into the sleeve 111 but she can still move her hand. Sleeve 111 that holds the arm is sewn to the bandage of the third body panel 105 of the main body panel 100 on the body core so it only allows you to move the arm minimally away (out, up and/down) from the body where a sling does not limit the arm from moving it from your body.

FIG. 1B is a perspective view of the arm restricting bandage of FIG. 1A in an overlapping position of the loop material disposed over the vertical strip. Main body panel 102 is in an overlapping position where the left end 101a left end 103 and left end 105a include the vertical strip 107. Vertical strip 107 may be referred to as a first closure mechanism 107. (FIG. 1A). In another embodiment of the invention, the vertical strip 107 may be a horizontal strip 107 of a hook material. The right end 101b, right end 103b and right end 105b include the loop material 109. (FIG. 1A). Loop material may also be referred to as a second closure mechanism 109. When the vertical strip 107 is connected with the loop material 109 in the overlapping position it becomes a hook and loop fastener such as the trademark VELCRO used to secure the main body panel 102 onto a torso 117 of a person 115 (FIG. 1C). VELCRO is a trademark of Velcro USA, located at 406 Brown Avenue, Manchester, N.H. 03103. In another embodiment of the invention, any type of conventional fasteners may be used in place of the vertical strip 107 and the loop material 109.

FIG. 1C is an embodiment of the arm restricting bandage of FIG. 1B wrapped around a torso of a person in accordance with the invention. A person 115 has the arm restricting badge 100 wrapped around her torso 117 onto the side of the person 115. Person 115 would be an adult that can adjust the hook 107 and loop 109 material. Person 115 would be able to place her arm 119 consisting of her hands, wrists and forearm through the opening 111a of sleeve 111 where the hands, wrists and forearm would be immobilized in the sleeve 111.

FIG. 1D is an embodiment of the arm restricting bandage of FIG. 1B showing a back portion wrapped around a torso of a person in accordance with the invention. Person 115 has the arm restricting bandage 100 wrapped around her torso 117. The person 115 would be a child who would need another person to put the hook 107 on top of the loop 109. On a back 121 of the person the hook 107 and 109 loop feature is shown. Another person adjusts the hook 107 and loop 109 to comfortably fit around her torso 117.

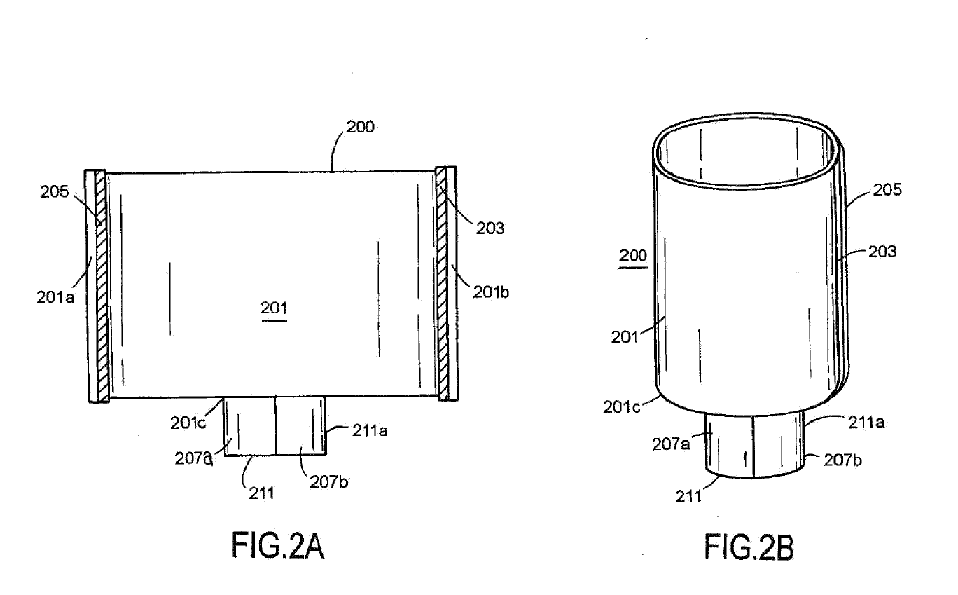

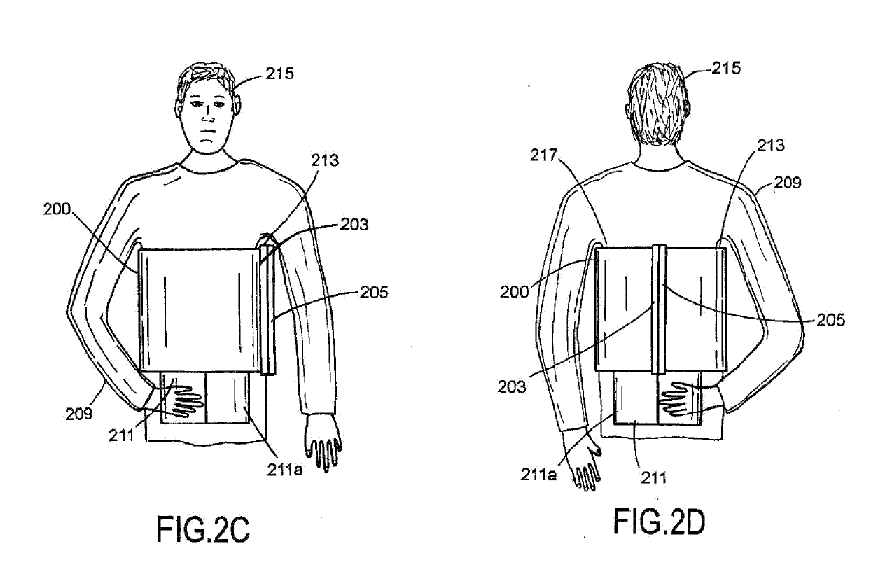

FIG. 2A is a perspective view of another arm restricting bandage in a laid open position in accordance with the invention. The arm restricting bandage 200 has a main body panel 201 that has generally elongated rectangular shape when laid open as shown. In another embodiment of the invention, the main body panel 201 may have any shape such as a square shape, circular shape, triangular shape, elliptical shape or any type of shape. The main body panel 201 may be made of any suitable elastic or non-elastic, breathable material, such as an ACE bandage. The main body panel 102 may be used for body core stabilization. Main body panel 201 has a length in the range of 12-40 inches and a width in the range of 5-20 inches. Left end 201a includes a vertical strip 205. Right end 201b includes a loop material 203. The main body panel 201 has a left end 201a, a right end 201b, and a bottom end 201c. A sleeve 211 is connected to the bottom end 201c where the sleeve 211 is stitched to the bottom end 201c of the main body panel 201. The sleeve 211 has an opening 211a that goes through the entire sleeve 211 where a person can insert his or her arm including hands, wrist and forearm through the sleeve 211. Opening 211a has range of 4-15 inches circumferentially from the bottom end 201c to fit the hand, wrist and the forearm In addition, this sleeve 211 includes a first portion 207a and a second portion 207b consisting of elastic breathable bandages that are circularly connected to the bottom end 201c to form the opening 211a. First portion 207a and the second portion 207b have a total length in a range of 6-16 inches to fit the hand, wrist and the forearm. Sleeve 211 that holds the arm is sewn to the bandage of the main body panel 201 on the body core so it only allows you to move the arm minimally away (out, up and/down) from the body where a sling does not limit the arm from moving it from your body.

FIG. 2B is a perspective view of the arm restricting bandage of FIG. 2A in an overlapping position of the loop material disposed over the vertical strip in accordance with the invention. Main body panel 201 is in a wrapped position where the left end 201a includes a vertical strip 205. (FIG. 2A). Vertical strip 205 may be referred to as a first closure mechanism 205. In another embodiment of the invention, the vertical strip 205 may be a horizontal strip 205 or a hook material. The right end 201b includes a loop material 203. (FIG. 2A). Loop material may also be referred to as a second closure mechanism 203. When the vertical strip 205 is connected with the loop material 203 it becomes a hook and loop fastener such as the trademark VELCRO used to secure the main body panel onto a torso of a person. In another embodiment of the invention, any type of conventional fasteners may be used in place of the vertical strip 205 and the loop material 203. As stated above, sleeve 211 includes the first portion 207a and the second portion 207b consisting of elastic breathable bandages that are circularly connected to the bottom end 201c to form the opening 211a.

FIG. 2C is an embodiment of the arm restricting bandage of FIG. 2B wrapped around a torso of a person in accordance with the invention. A person 215 has the arm restricting badge 200 wrapped around her torso 213. The person 215 would be able to place her arm 209 consisting of her hands, wrists and forearm through the opening 211a of sleeve 211 where the hands, wrists and forearm would be immobilized in the sleeve 211. Also, the person 215 would be an adult that adjusts the hook 205 and the loop 203 material to comfortably fit on her torso 213.

FIG. 2D is an embodiment of the arm restricting bandage of FIG. 2B showing a back portion wrapped around a torso of a person. Person 215 has the arm restricting bandage 200 wrapped around her torso 213. On the back 217 of the person 215 the hook 205 and loop 203 feature is shown. Another person would be able to adjust the hook 205 and loop 203 to comfortably fit around the torso 213 of the person 215. The person 215 would be able to place her arm 209 consisting of her hands, wrists and forearm through the opening 211a of the sleeve 211 where the hands, wrists and forearm would be immobilized in the sleeve 211.

This invention provides a simple and effective bandage and method to immobilize an arm of a person, such as the person's hand, wrist and forearm. The bandage allows a person to simply immobilize a portion of his or her body that does not allow large movement of the arm away from the body. In addition, this bandage prevents a child from slipping her arm out of the bandage herself, while not causing his or her neck to be out of alignment. The person is able to utilize a flexible arm restricting bandage that is elastic, breathable and flexible so she can accomplish normal everyday tasks while still keeping her arm immobilized sufficient for it to heal properly.

Although the present invention has been described above in terms of specific embodiments, many modifications and variations of this invention can be made as will be obvious to those of ordinary skill in the art, without departing from its spirit and scope as set forth in the following claims.

What is claimed is:

1. A bandage for restricting movement of an arm of a person, comprising:
at least one body panel formed of an elastic breathable material, wherein he at least one body panel has a left end, a right end and a bottom end, wherein the left end has a first closure mechanism, wherein the right end has a second closure mechanism;
the at least one body panel is configured to wrap around a torso of the person, wherein the first closure mechanism and the second closure mechanism connects to each other to hold the at least one body panel onto the torso of the person; and
a sleeve formed of an elastic breathable material coupled to the bottom end of the at least one body panel, wherein the sleeve is configured to receive an arm of the person, wherein the arm is held in place by the sleeve.

2. The bandage of claim 1, wherein the first closure mechanism is a loop material.

3. The bandage of claim 2, wherein the second closure mechanism is a hook material.

4. The bandage of claim 1, wherein the at least one body panel has a length in a range of 12-40 inches.

5. The bandage of claim 1, wherein the at least one body panel has a width in a range of 5-70 inches.

6. The bandage of claim 1, wherein the at least one body panel is an elastic breathable bandage.

7. A bandage for restricting movement of an art of a person, comprising;
a plurality of body panels formed of an elastic breathable material, wherein each of the plurality of body panels have a left end, a right end and a bottom end, wherein the left end and the right end have fastening members to connect the left end to the right end;
the plurality of body panels are configured to wrap around a torso of the person, wherein the fastening members hold the plurality of body panels onto the torso of the person; and
a sleeve formed of an elastic breathable material coupled to the bottom end of one of he plurality of body panels, wherein the sleeve is configured to receive an arm of the person, wherein the arm is held in place by the sleeve.

8. The arm restricting bandage of claim 7, wherein the fastening members are a hook and loop feature.
